(19) United States
(12) Patent Application Publication
(10) Pub. No.: US 2019/0350208 A1
(43) Pub. Date: Nov. 21, 2019

(54) Activators of Plant Metabolic Changes

(71) Applicants: Université de Mons, Mons (BE); Materia Nova ASBL, Mons (BE)

(72) Inventors: Juan Carlos Cabrera, Liège (BE); Ruddy Wattiez, Sirault (BE)

(21) Appl. No.: 16/475,816

(22) PCT Filed: Jan. 3, 2018
(86) PCT No.: PCT/EP2018/050147

§ 371 Date: Jul. 3, 2019

(30) Foreign Application Priority Data
Jan. 3, 2017 (EP) 17150194.3
Jan. 3, 2017 (EP) PCT/EP2017/050094

(51) Int. Cl. A01N 63/02; A01N 43/16; A61K 31/7004

(52) U.S. Cl. CPC A01N 63/02; A61K 31/7004; A01N 43/16

(57) ABSTRACT

The present invention relates to a composition comprising saccharides extracted from blue-green algae biomass, the said saccharides comprising—from 55% to 60% of rhamnose by weight, —from 6.5% to 10% of uronic acid by weight, —from 5% to 15% of sulfate groups by weight, and—less than 10% of xylose, glucose and galactose or a mixture thereof calculated by weight upon the total of the composition being 100%.

### FIELD OF THE INVENTION

The present invention is in the field of agronomy, especially in the protection of plants and is related to a composition extracted from blue-green algae, possibly combined with other (preferably polycationic) molecules, especially with (polycationic) saccharides, that improves the positive plant metabolic changes and agronomic properties of this composition.

The present application is also related to the use of this composition based on derived (poly-anionic saccharides and alcohol soluble compositions extracted from blue-green algae for induction of positive plant metabolic changes.

The present application is related more particularly to the use of this composition based on derived (poly) saccharides and alcohol soluble compositions extracted from blue-green algae, possibly combined with others molecules especially with saccharides in the domain of pharmacy, especially in wound healing, cosmetology and for nutraceutics production.

### STATE OF THE ART

Use of chemical fertilizers and pesticides and its associated natural habitat destruction has caused a “major extinction event” and experts predict the trend to continue, thus lowering the world's biodiversity and changing its ecology. In this dramatic way, attention to environmental impact of known or new chemical compounds in agriculture should be done, especially because world population is expected to double the present population by the year 2050 and the conversion of large natural ecosystems, will require farmers to produce food doubles.

One of the most imperative hitches concerning the production of food crops is the difficulty of controlling plant diseases to maintain the high quality and yield, which the producer and consumer expect. In addition, numerous plant pathogens have developed resistance to the active ingredients of a wide range of agrochemicals causing systematic loss of broadly used pesticides from the market.

Presently, one of the most pioneering approach to the control of plant diseases is through the enhancement of the plant's own defence mechanisms (induced resistance), which would not necessarily involve the application of toxic compounds to plants. It is very well established for over 100 years, that plants can defend themselves. However, in the last 20 years, a significant progress in our knowledge on plant immunity, has provided the understanding necessary to allow induced resistance to be used in practice.

Spirulina (Arthrospira platensis) is a multicellular, filamentous cyanobacterium belonging to a blue-green alga of cyanobacteria. Spirulina is one of the edible microalgae that has been successfully produced and is in widespread use. This blue green alga grows naturally in warm climate countries and has been considered as supplement in human and animal food, since it can be employed as a source of valuable proteins, vitamins, amino acids, minerals, fatty acids, etc. Spirulina derived compounds are also used in cosmetic industry, and during the last years an increasing number of preclinical and clinical studies also suggest certain pharmacological and therapeutic effects (Gershwin M E, Belay A (eds) (2008) Spirulina in human nutrition and health. CRC Press, Boca Raton, 312 pp).

(Poly) saccharides from Spirulina have been found to possess a variety of therapeutic activities including antiviral activity against herpes simplex virus type 1 (HSV-1) (N. Chirasuwan et al, Kasetsart Journal (Natural Science) 41 (2007) 311-318), inhibitory effects on corneal neovascularization (L. Yang et al, Molecular Vision 15 (2009) 1951-1961), anticoagulant activity mediated by heparin cofactor II (H. Majdoub, B. M. et al, Biochimica et Biophysica Acta 1790 (2009) 1377-1381); and also antioxidant capacity (Chaiklahan et al. International Journal of Biological Macromolecules 58 (2013) 73-78). Polysaccharides extracted from spirulina biomass are referred as “spirulan”. Therefore, it is known that several extracts or molecules obtained from Spirulina may find application in cosmetic, food and pharmaceutical industries.

European Patent application EP 1 729 582 discloses the use of ulvans or of ulvan-derived oligosaccharides obtained by acid hydrolysis or enzymatic hydrolysis of said ulvans, as activators of plant defence and resistance reactions against biotic or abiotic stresses. In EP 1 729 582, these ulvans are extracted from the marine macroalgae of the genus ulva or Enteromorpha species. Ulvans are sulphated polysaccharides structurally different than others polysaccharides from green, brown and red seaweeds macroalgae. However, until now it has not yet described that specific extracts, especially (poly) saccharides and methanol soluble compounds extracted from Spirulina, may induce plant defense reactions and others metabolic changes dealing with plant resistance/tolerance to biotic and abiotic stresses.

### AIMS OF THE INVENTION

The present invention aims to obtain new composition and its use that do not present the drawbacks of the products and methods of the state of the art, especially a new elicitor or bio-stimulating composition made of naturals products, obtainable from a natural renewable source and comprising bio-stimulants or activators of metabolites that finds improved properties in various applications in the agricultural domain.

Another aim of the present invention concerns the use of such composition or a method comprising the administration of this composition to a plant or a plant soil for the induction of plant metabolic changes, such as activation of the plants immune response or for increasing the protein content in plants.

A further aim of the invention is related to improved use of such composition in others technological fields, including paper production, pharmacology, especially in wound healing, cosmetology and/or nutraceutics production.

### SUMMARY OF THE INVENTION

The present invention is related to a composition, preferably made of natural products and obtainable from a non-expensive renewable source and comprising one or more bio-stimulants or activators of metabolites, these bio-stimulants or activators or elicitors, being (preferably derived from) poly-anionic(oligo) saccharides or poly-anionic polysaccharides extracted from blue-green algae, preferably from genus Spirulina, these bio-stimulants being possibly also combined with others bioactive poly-cationic molecules, such as chitosan saccharides, that finds improved characteristics in various applications in the agricultural domain. In the composition of the invention the saccharides, preferably the polysaccharides are selected from the group consisting of rhamnose accounting for (about) 55% in weight (% wt) to (about) 60% in weight (% wt) of the total saccharides, uronic acids accounting between (about) 6.5% in weight (% wt) to (about) 10% in weight (% wt) and one or more sulfate group(s) accounting between (about) 5% in weight (% wt) to (about) 15% in weight (% wt). Others saccharides such as xylose, glucose and galactose are present, but accounting for less than 10% in weight (% wt). All these % are calculated upon the total weight of all sugars or saccharides being 100%.

The present application is related to the use of this composition comprising these derived Spirulina poly-anionic saccharides, preferably these poly-anionic oligo or polysaccharides extracted from genus Spirulina from blue-green algae, possibly combined with other bioactive poly-cationic compounds, such as chitosan saccharides and their salts, for an induction of plant metabolic changes, such as activation of the immune response of plants and preferably improved resistance to diseases and/or for increasing protein content in plants, but also general yielding of the plant characteristics, such as an increased height, an increased thickness of their stem, leaves, fruits or roots, an increased biomass or an increased number of flowers and/or fruits per plant.

The preferred poly-cationic compounds present a synergy with the poly-anionic compounds, especially the poly-anionic oligo or poly-saccharides present in the claimed composition of the invention in the induction of plant metabolic changes as above described, in others suitable fields or to increase the known properties of the bioactive poly-cationic compounds. This addition allows also to reduce their possible side effects, particularly their toxicity involved, among others, in the cell death at high concentration levels of bioactive poly-cationic compounds, selected from the group of chitosan or functionally-modified chitosan and their salts, preferably their chloride, acetate, glutamate or lactate salts. This synergy is advantageously obtained not only in the induction of plant metabolic changes, but also in other fields, such as medical, pharmaceutical, neutraceutical, food, feed, cosmetic, environmental or industrial applications, especially in textile or paper production.

The term “chitosan” refers to a linear polysaccharide composed of randomly distributed Beta-(1, 4)-linked D-glucosamine (deacetylated unit) and N-acetyl-D-glucosamine (acetylated unit). Chitosan may have a molecular weight ranging from (about) 1 kDa to (about) 500 kDa.

The functionally-modified chitosan saccharides are preferably selected from the group consisting of N-[(2-hydroxy-3-trimethylammonium)propyl]chitosan (HTC) identified as CAS 106602-18-0, N-trimethyl chitosan (TMC), N,O-carboxymethyl chitosan (N,O-CMC), N-carboxymethyl chitosan (N-CMC), N,N-Carboxymethyl chitosan (N,N-CMC), O-carboxymethyl chitosan (O-CMC), hydrophobically-modified chitosan (HMC) and their salts, preferably their chloride, acetate, glutamate or lactate salts. Characteristics of these functionally-modified chitosan saccharides and their preparation methods are mentioned and described in the international patent application WO2015/055796 incorporated by reference.

In the composition of the invention, the chitosan degree of acetylation may be from (about) 1% to (about) 100%. Preferably the chitosan degree of acetylation is higher than (about) 5% and lower than (about) 50%. The degree of acetylation may be measured by 1H-NMR, as known in the art.

Others examples of preferred poly-cationic compounds are polysaccharides or oligosaccharides or functionally-modified saccharides, more preferably compounds selected from the group consisting of dextran or functionally modified dextran, such as hydrophobically-modified dextran (HMD), starch or functionally-modified starch, such as hydroxypropyl starch, amylose or functionally-modified amylose, amylopectin or functionally modified amylopectin, cellulose or functionally-modified cellulose, such as methylcellulose, carboxymethylcellulose, ethylcellulose, hydroxyethylcellulose, hydroxypropylcellulose, hypromellose, hypromellose acetate succinate, hypromellose phthalate, croscarmellose, cyclodextrin, dextrate, maltodextrin, pullulan, guar gum and their salts, preferably their chloride, acetate, glutamate or lactate salts or a mixture thereof.

A preferred aspect of the invention concerns also a hydrolysate, preferably a enzymatic hydrolysate, more preferably a pectinase hydrolysate of the polysaccharide composition according to the invention, preferably an hydrolysate comprising oligo saccharides of lower molecular weight than the treated polysaccharides according to the invention and made of oligosaccharides having a low molecular weight, preferably a molecular weight comprised between (about) 300 KD and (2500) KD. This hydrolysate is preferably obtained by an incubation of the polysaccharide solution according to the invention containing the composition of the invention with a suitable amount of one or more hydrolase enzyme(s), preferably selected from the group consisting of xylanases, cellulases or pectinases, at suitable pH and temperature and during a suitable time.

The present composition comprising the bio-stimulants or activators or elicitors according to the invention may also comprise or may be related to the alcohol soluble, preferably an ethanol (ETOH) or a methanol (METOH) soluble, more preferably a 30% ethanol (30% ETOH) or a 30% methanol (30% METOH), fraction extracted from Spirulina and comprising beta-carotenes and fatty acids, preferably C16 fatty acids, C18 fatty acids, their salts or a mixture thereof. These different (polysaccharide, oligosaccharide and alcohol soluble) fractions isolated from Spirulina presenting advantageously a synergic effect in the induction of plant metabolic changes. The present composition(s) of the invention may also comprise one or more other additional (stimulant or activator) element(s) selected from the group consisting of growth regulators or growth factors, minerals, ions, nutriments, food or feed additive, flavourings, colours, vitamins and fragrances for improving the mentioned properties of the composition(s) according to the invention.

The present application is related more particularly to the use of the composition(s) of the invention and comprising these derived poly-anionic saccharides, preferably these poly-anionic oligo or polysaccharides extracted from blue-green algae, especially from genus Spirulina (spirulina saccharides), possibly combined with others bioactive poly-cationic compounds, such as chitosan saccharides, functional-modified chitosan saccharides or their salts, but also the alcohol soluble fraction(s) of the invention in the domains of pharmacy, cosmetology or nutraceutics production.

The composition(s) (comprising the poly-anionic saccharides or the alcohol soluble fractions) according to the invention, preferably further comprising the chitosan saccharides or functionally-modified chitosan saccharides or their salts, can be used in the beverage and food or feed technologies, for recovering from beverages, organic particles, microorganisms, colloids, proteins, heavy metals, residual pesticides, mycotoxins and endotoxins from different liquid medium such as beer, milk or wine.

The composition(s) according to the invention, preferably comprising the chitosan saccharides, the functional-modified chitosan saccharides or their salts, can be used as natural food additive, for obtaining anti-microbial and anti-fungal activities against a wide range of fungi, including yeast, and bacteria and can also be used as adjuvant for conventional food preservative and anti-browning agents, as component for gas permeable edible films suitable for fruits/vegetable storage, as thickening agent, as stabilizing agent, as emulsifying agent, as thixotropic agent or as natural flavour extender.

The anti-bacterial and anti-fungal activities of the composition(s) according to the invention, preferably comprising the chitosan saccharides, the functional-modified chitosan saccharides or their salts, can also be used in food or feed industry for obtaining extended shelf life, delay ripening and decay of fruits or vegetables, for the preservation of a food or feed selected from the group consisting of meat, crustacean, oysters, fruits or vegetables and finished product, possibly in combination with conventional preservatives, preferably with sulphite or sodium benzoate, or as an alternative to these preservatives.

The composition(s) according to the invention, preferably with the chitosan saccharides, the functional-modified chitosan saccharides or their salts can also be applied upon textile fibres in the form of a film or by impregnation of these fibres or tissue with a solution comprising the composition of the invention. Therefore the property of the fibres of a textile may be modified through its improved anti-fungal or anti-bacterial properties. This textile may also corresponds to a medical textile used for the treatment of wounds.

The cosmetic applications of the composition(s) according to the invention, preferably comprising the chitosan saccharides, the functional-modified chitosan saccharides or their salts, are suitable for skincare formulation in the form of the cream or a lotion, or for hair care formulation as sprays or shampoos, in make-up composition or in tooth pastes and for their anti-UV properties, in the preparation of deodorants, in compositions of oral hygiene and in compositions for the encapsulation of pigments. Advantageously, due to its non-animal origin, it is possible to use the composition according to the invention, without inducing the risk of allergy.

In environmental applications, the composition(s) according to the invention, preferably comprising the chitosan saccharides, the functional modified chitosan saccharides or their salts, could be used as a chelating agents of heavy metals, for the treatment of waste water, especially in water purification, for the segregation of organic compounds and heavy metals, for precipitating waste compound of other compounds, like DDT and polychlorobenzenes or for fixing radicals.

The composition(s) according to the invention, preferably comprising the chitosan saccharides, the functionally-modified chitosan saccharides or their salts, could also be used in manufacturing process of paper, to replace some amino substituents, such as gum or polysynthetic polysaccharides and to reduce the use of chemical additives.

It is also possible that the paper obtained by the use of the composition(s) according to the invention, may present a smoother surface and better resistance to moisture; but such composition(s) can also be used for the production of sanitary paper, packing paper and paperboard.

Another application of the composition(s) according to the invention, preferably comprising the chitosan saccharides, the functionally-modified chitosan saccharides or their salts, is in the field of medicine and pharmaceutical applications. This composition could be applied as anti-adhesive surgical aid, to prevent adhesion between tissues during surgery and as adjuvant for vaccines.

Therefore, the present invention is also related to any biomaterial which comprises the composition(s) according to the invention, preferably with the chitosan saccharides, the functionally-modified chitosan saccharides or their salts, for specific medical, diagnostic or research applications of compounds to be released in the intestines, due to non-digestion of the compositions of the invention in the patient stomach, for instance through a delayed release of an active compound present in the composition(s) of the invention. Its muco-adhesive properties can also be used for obtaining a good contact with skin layer of for improving innovative drugs delivery system through local and systemic administration, for the production of tablets, as wetting and coating agents, to form complexes with anionic, cationic or amphiphilic drugs, for improving the solubility of poorly water soluble drugs, for enhancing absorption of drugs across mucosal tissues and for potentiating immunological response of vaccines, preferably as adjuvant of vaccines.

The known properties of the chitosan saccharides, the functionally-modified chitosan saccharides or their salts, especially in wound healing, allows the use of the composition(s) of the invention, comprising the chitosan saccharides, to treat the formation of fibrin bits in wounds, to prevent the formation of scars and to support cell regeneration.

The composition(s) of the invention can be also useful applied for tissue regeneration, cell transplantation, regeneration and encapsulation in air-permeable films, to form sutures and bandages, for the manufacturing of artificial skin, for the reconstruction of tissues and organs.

In these pharmaceutical applications, the elements of the composition(s) according to the invention, can be combined with known compounds used in tissue or organ repairs, such as bone or cartilage, particles, ions, salts, growth factors and hormones, plasma derivatives, preferably selected from the group consisting of one or more coagulation factor, fibrinogen, platelets, antibiotics, hormones, vitamins, cytokines, interferons, proteins, including cartilages, elastin, fibrin, their precursors or a mixture thereof.

The composition(s) according to the invention could be presented in various forms, preferably a form selected from the group consisting of a liquid form, such as aqueous solution, particles form like nanoparticles, microspheres, pellets, suspension or emulsion, as well as porous or semi-porous form with suitable carriers, like methyl cellulose or gums.

The composition(s) of the invention can be applied to growing crops, as a preservative coating and biostatic agent, upon the plant leaves, the plant roots, the plant seeds or the soil of the plants with suitable carriers used in the agriculture, like stabilizer or buffer material. Each amount of solution of solid compositions and its carrier is adapted by the skilled person for each type of crop to be treated and protected. This amounts may vary according to the part of the plant to be treated, the climate, the season period and day time of administration. The composition(s) according to the invention are preferably included in a (preferably an aqueous or an alcohol) solution at a concentration higher than 0.1 m/ml, preferably ata concentration comprised between (about) 0.1 mg/ml and (about) 10 mg/ml, this values depend upon the administration mode (plant suspension in a solution comprising the composition or direct administration of the solution upon the plant soil, the plant leaves or the plant roots, but also an efficient effect upon the physiology plant or upon the plant soil characteristics.

The concerned plants are dicots and monocots, preferably economically important plants, more preferably plants or plant parts, such as plant roots, plant leaves, plant fruits, plant calluses, or plant seeds, that are preferably selected from the group consisting of tomato, carrot, cucumber, pea, lettuce, capsicum, beet, sugar beet, potato, wheat, corn, rice, barley, cotton, sunflower, Soybean, peanut, bean, chicory, sprout, cauliflower, broccoli, radish, spinach, Eggplant, onions, garlic, pepper, celery, apple, pear, cherry, melon, lemon, lime, pomelo, orange, kiwi, papaya, citrus, strawberry, pineapple, tea, coffee, tobacco, grape fruit, papaya, mango, passion fruit, Banana, avocado, almond, vine, olive tree, soybean, sugarcane and ornamental plants, trees and flowers.

### Definitions

A used herein, the singular forms “a”, “an” and “the” include both singular and plural referents unless the context clearly indicates otherwise. The term “about” used herein, when referring to a measurable value, such as a parameter, an amount, a temporal duration and the like, is meant to encompass variations of and from the specified value, in particular variations of +/−10% or less, preferably +/−5% or less of and from the specified value, insofar such variations are appropriate to perform in the context of the disclosed invention.

Whereas the terms “one or more”, such as one or more members of a group of members, is clear per se, by means of further exemplifications, the terms encompass a reference to any one of said members, or any of two or more or of any of three or more members of said members and up to all of said members. The term “compositions” refer to the poly-anionic polysaccharides extract composition obtained from blue-green algae, especially extracted from Spirulina, this extract being possibly combined with the poly-cationic compound above described, the enzymatic hydrolysate of this poly-anionic polysaccharide composition comprising oligosaccharides of lower molecular weight in such composition and the above described alcohol soluble fraction extracted from Spirulina

The term “saccharides” or “carbohydrates” refers to an organic compound comprising only of carbons, hydrogens and oxygens. Non limiting examples of saccharides or carbohydrates are oligosaccharides or polysaccharides. The term “polysaccharide” refers to a polymer or macromolecule consisting of monosaccharide units joined together by glycoside bonds. Polysaccharides may be linear or branched. The “polysaccharide” may comprises from (about) 20 to about (5000) or more monosaccharides units, preferably from (about) 40 to (about) 2500 monosaccharide units. The “oligosaccharide” comprises from 2 monosaccharide units to (about) 20 monosaccharides units joined by glycosidic linkages.

The term “functionally-modified saccharide”, refers to a saccharide, wherein one or more of its functional groups are chemically modified, preferably wherein units or groups are modified to, replaced by, or substituted by others groups or units, preferably by addition of hydroxyl groups (in glucose or galactose units) or amine groups in glucosamine units or galactosamine units, such as for example to alter one or more of the chemical or physical properties of the saccharide, for instance to increase or decrease the hydrophobicity (physical property of a molecule to be repelled from a mass of water) of the saccharide.

The term “rhamnose” corresponds to a naturally occurring deoxy sugar, classified as a methyl pentose or a 6-deoxy-hexose (IUPAC name being: (2R, 3R, 4R, 5R, 6S)-6-Methyloxasne-2, 3², 4, 5-tetrol).

The term “uronic acid” is a class of sugar acids with both carbonyl and carboxylic acid functional groups. They are sugars in which the terminal carbon hydroxyl group has been oxidized to a carboxylic acid.

The terms “fatty acid(s)” refer to a carboxylic acid with a saturated or unsaturated aliphatic chain of carbon atoms.

The terms “sulphated group(s)” correspond to group(s) comprising S=0 or C—O—S radicals.

The terms “treat” or “treatment” encompass both the therapeutic treatment of an already disease or condition or syndrome, such as therapy of an already developed diseases as well as prophylactic or preventive measures, wherein the aim is to prevent or lessen the chances of incidence of undesired affliction, such as to prevent occurrence, development and progression of specific described diseases, their symptoms, side effects or consequences. Treatment may also correspond to prolonging survival as compared to expected survival, if not receiving treatment.

The terms “bio-stimulant(s)”, “activator(s)” or “elicitor(s)” are defined as compounds which activate chemical defence in plants and which are able to trigger immune defence responses in plants. Preferably, these compounds are non-synthetic, i.e. natural molecules present in nature, preferably in plants, animals or microbial flora and interacting with treated plants, through various biosynthetic pathways that are activated in these treated plants depending on the compound(s) used. Elicitation can be explained as mechanism(s) by which cells and tissues of organisms respond and adept to changes in external environmental conditions. In many cases, these mechanisms use pathways of specific receptors for particular chemicals. This binding triggers a cascade of events within the cells, including up-regulation or down-regulation of genes or transcriptional factors, as well as activation or repression of specific pathways within the cells, including substantial changes in the cellular physiology. An example of elicitor-based activity includes induction of immune or resistance response in plants.

According to the European Bio-stimulant Industry Council (EBIC), plant bio-stimulants contain substance(s) and/or micro-organisms whose function when applied to plants or the rhizosphere is to stimulate natural processes to enhance/benefit nutrient uptake, nutrient efficiency, tolerance to abiotic stress, and crop quality. Bio-stimulants have no direct action against pests, and therefore do not fall within the regulatory framework of pesticides.

According to the US Environmental Protection Agency (EPA), bio-pesticides relate to pesticides derived from such natural materials as animals, plants, bacteria and certain minerals. For example, canola oil and baking soda have pesticide applications and are considered as bio-pesticides. As of April 2016, about 300 registered bio-pesticide active ingredients and about 1400 active bio-pesticide product registrations exist.

Bio-pesticides fall into three major classes:
1. Biochemical pesticides that are naturally occurring substances used to control pests by non-toxic mechanisms. Conventional pesticides, by contrast, are generally synthetic materials that directly kill or inactivate the pest. Biochemical pesticides include substances that interfere with mating, such as insect sex pheromones, as well as various scented plant extracts that attract insect pests to traps. Because it is sometimes difficult to determine whether a substance meets the criteria for classification as a biochemical pesticide, EPA has established a special committee to make such decisions.
2. Microbial pesticides consist of a microorganism (e.g., a bacterium, fungus, virus or protozoan) as the active ingredient. Microbial pesticides can control many different kinds of pests, although each separate active ingredient is relatively specific for its target pests. For example, there are fungi that control certain weeds and other fungi that kill specific insects.
   The most widely used microbial pesticides are subspecies and strains of Bacillus thuringiensis (or Bt.) Each strain of this bacterium produces a different mix of proteins and specifically kills one or a few related species of insect larvae. While some Bt. ingredients control moth larvae found on plants, other Bt. ingredients are specific for larvae of flies and mosquitoes. The target insect species are determined by whether the particular Bt. produces a protein that can bind to a larval gut receptor, thereby causing the insect larvae to starve.
3. Plant-Incorporated-Protectants (PIPs) are pesticide substances that plants produce from genetic material that has been added to the plant. For example, scientists can take the gene for the Bt. pesticide protein and introduce the gene into the plant's own genetic material. Then the plant, instead of the Bt. bacterium, manufactures the substance that destroys the pest. The protein and its genetic material, but not the plant itself, are regulated by EPA.

According to the Belgian Legislation, bio-pesticides are a sub-group of products derived from natural materials in plant protection products. Bio-pesticides meet the definition of plant protection product as filled in Article 2 of (EC) 1107/2009.

Bio-pesticides can be divided into 4 major categories of products.
1. Products based on plant extracts
   It is a wide range of plant protection products of various kinds. This products set is between non-processed plants extracts products to products undergoing multiple transformations. Some non-limitative examples of such products are azadirachtin, pyrethrine or vegetable oils.
2. Products containing a microorganism
   In this products set are grouped living products, such as microorganisms selected from the group consisting of bacteria, viruses and fungi. Some non-limitative examples of these microorganisms are: Paecilomyces fumosoroseus or Bacillus thuringiensis.
3. Pheromones
   Pheromones are chemicals released by plants and animals that change the behavior of other individuals within the same species.
   Pheromones are subject to authorization for the following fight methods:
      “mating disruption” which is an excess dispersion in the environment of a sex pheromone so that the males are unable to locate females.
      “mass trapping” which is a rapping technique to attract the insect population to a source of pheromones to capture it.
   Pheromones present in the following control methods should not be allowed:
      “Attract & Kill” which is a trapping technique combining pheromone and an insecticide. Pheromones used are considered co-formulating.
      “monitoring” which is a set of techniques whose objectives are to know the state of the density of an insect population on a given plot.
4. Other bio-pesticides
   Some products are not based on biological control agents, present a lower risk to the environment or human health. These products can be associated with bio-pesticides.

All documents cited in the present specification are hereby incorporated by reference.

The present invention will be described in details in the following examples in reference to the enclosed figures presented as non-limiting preferred embodiments of the invention.

### BRIEF DESCRIPTION OF THE DRAWINGS

FIG. 1 A represents the GC-MS chromatograms of the different XAD fractions and shows that the Spirulina alcohol extract comprises C16 and C18 fatty acids.

FIGS. 1 B and C represent respectfully the MS spectra of the XAD fraction in ethanol and in acetone.

FIG. 1.2 represents FTIR spectra and monosaccharide composition of Spirulina poly-anionic saccharides extract according to the invention.

FIG. 1.3 represents the thermogravimetric analysis of the Spirulina poly-anionic saccharides extract composition according to the invention.

FIG. 1.4 represents the relative viscosity of the (poly-anionicsaccharide extract solutions in function of the concentration.

FIG. 1.5 represents the Macro-Prep DEAE-chromatogram of spirulina (poly-anionic) saccharide extract solutions.

FIG. 2 represents the protein content and PAL activity in Arabidopsis thaliana cell suspension, 24 hours after treatments with increasing concentrations of Spirulina saccharides extract.

FIG. 3 shows that foliar spraying of the composition according to the invention increases protein production in the treated plant.

FIG. 4 represents the protein content and PAL activity in leaves of 10 days old wheat plants from seeds coated with increasing concentrations of Spirulina poly-anionic saccharides extract according to the invention.

FIG. 5 represents the protein content and PAL activity in leaves of 10 days old wheat plants growing on a soil pre-treated with a solution of Spirulina poly-anionic saccharides extract or Spirulina MeOH soluble extract.

FIG. 6.1 represents the protein content and PAL activity in leaves of tomato, wheat and cucumber plants from seedling pretreated with increasing concentrations of the spirulina poly-anionic saccharides extract and increasing dilution of spirulina MeOH extract according to the invention.

FIG. 6.2 represents the PAL activity in leaves of 10 days old tomato plants from seedling pre-treated by roots dipping with Spirulina poly-anionic saccharides extract according to the invention, chitosan or a combination of both.

FIG. 7 represents the protein and PAL activity in tomato leaves treated with spirulina poly-anionic saccharide extract alone or in combination with oligoglucan and chitooligosaccharides.

FIG. 8 represents the protein content and PAL relative activity obtained with increased concentrations of the added Spirulina poly-anionic saccharides fraction of the invention.

FIG. 9 represents the experimental steps applied in the examples

FIG. 10 represents the protein content and PAL relative activity according to increased concentration of added Spirulina poly-anionic saccharides fraction of the invention.

FIG. 11 represents the metabolite GC-MS analysis of the different hydrolysate fragments of the Spirulina poly-anionic saccharides fraction of the invention.

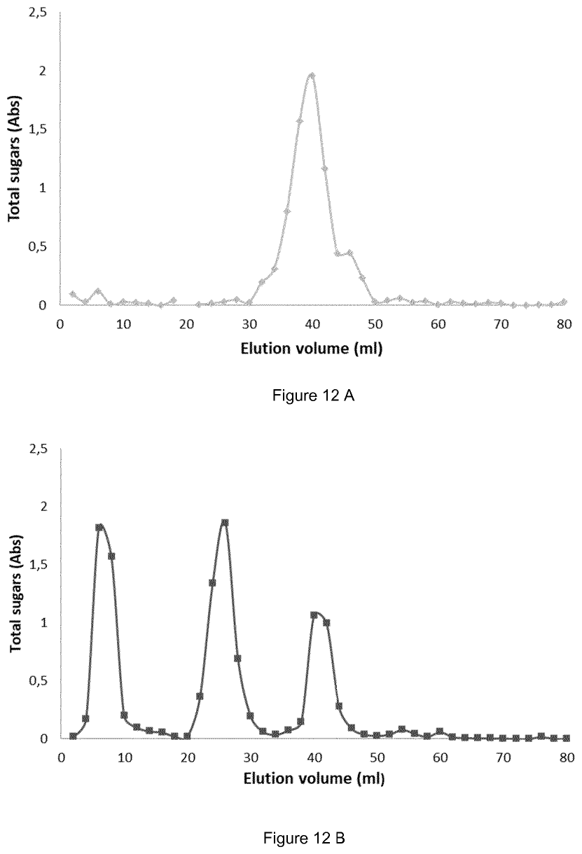

FIGS. 12A and 12B represents anion-exchange chromatographic profile of the hydrolysate fragments of the invention.

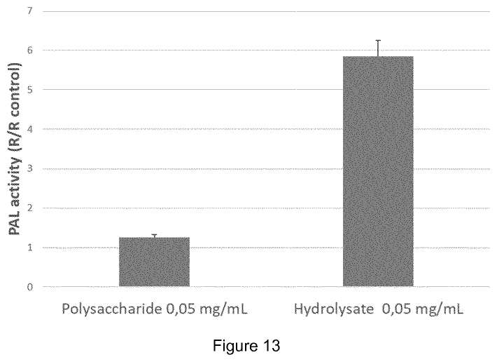

FIG. 13 represents the PAL relative activity obtained after addition of the poly-anionic saccharide extract and the fragments hydrolysate according to the invention.

FIG. 14 represents the production step of the different Spirulina ETOH fractions according to the invention.

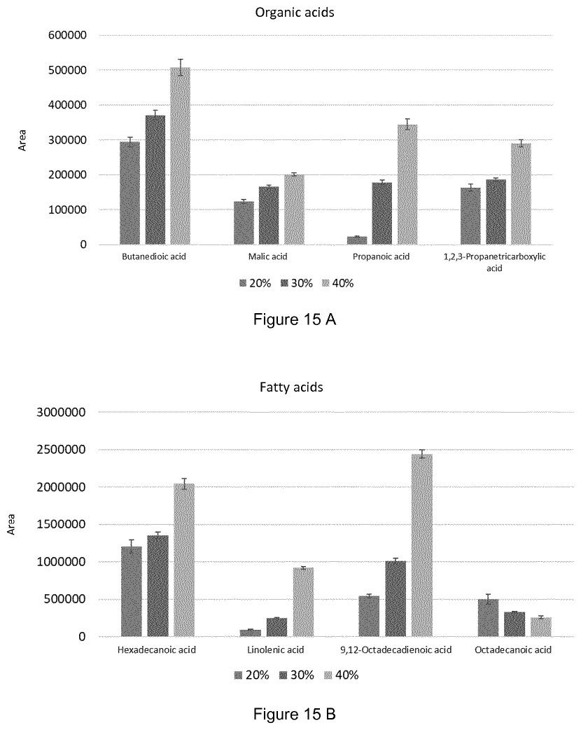

FIGS. 15A to 15C represent the bioactive molecules profile of the obtained ETOH fractions of the invention.

FIG. 16 represent the protein content and PAL relative activity after extraction with increased Ethanol percentages.

### DETAILED DESCRIPTION OF THE INVENTION

Two main fractions present in the composition according to the invention are obtained from a blue-green algae product, being Spirulina genus biomass: An alcohol, preferably a methanol soluble fraction and a (poly) saccharides fraction. This “biomass” being a cells pellet obtained after filtration of a Spirulina culture associated or not, to a drying step, such as lyophilisation or an air drying step. The measure of a plant biomass, which is basically the density or amount of plant-life, is also known to be directly related to crop yield and can be used to measure this crop yield.

In the Spirulina methanol (MEOH) and ethanol (ETOH) soluble fraction, fatty acids, preferably mostly the C16, C18 fatty acids, beta-carotenes and low molecular weight molecules are identified and show antimicrobial activity.

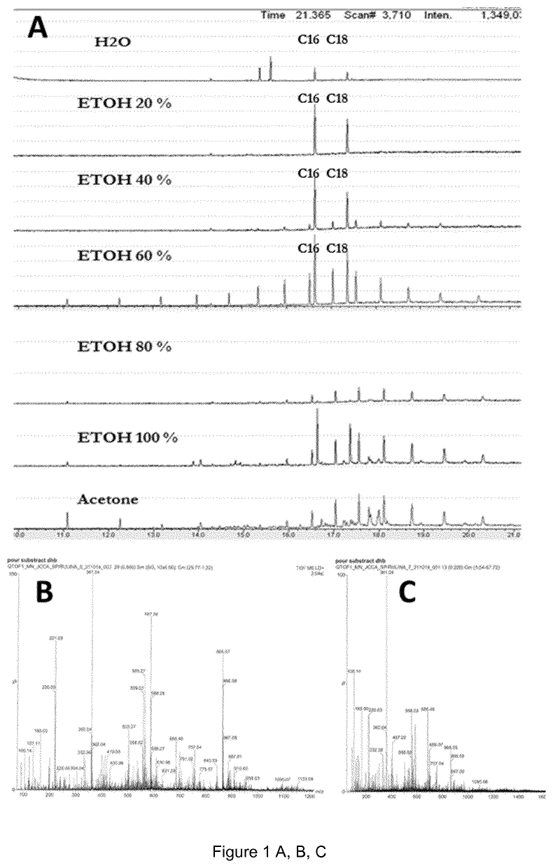

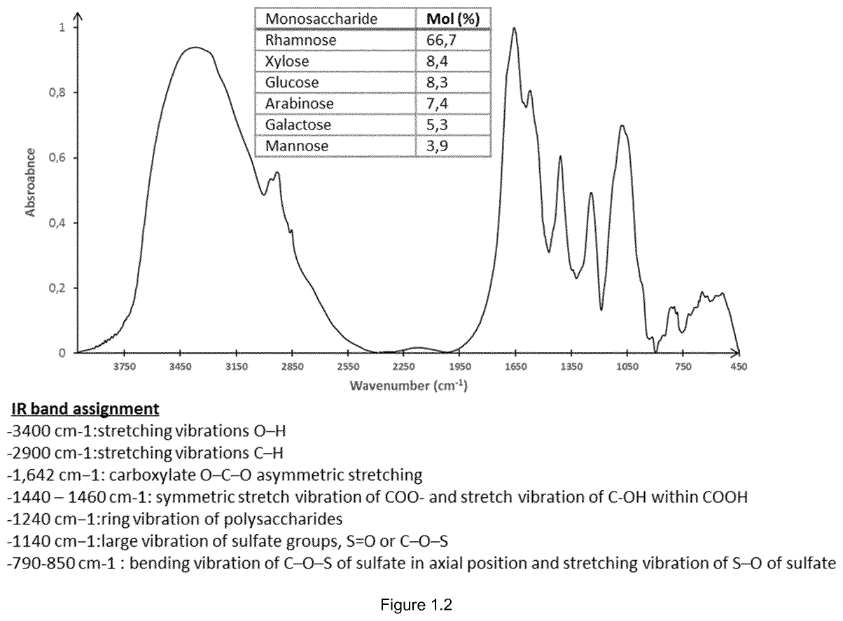

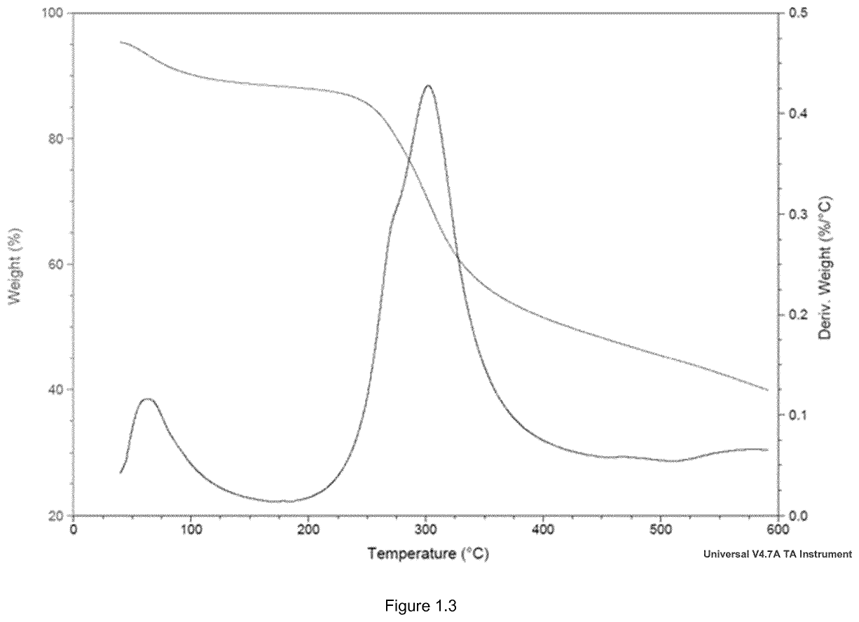

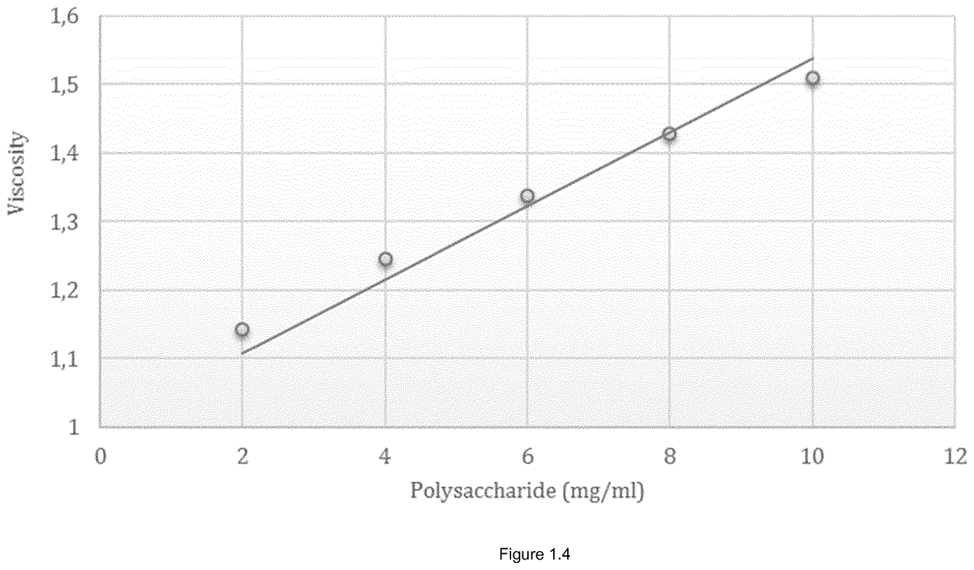

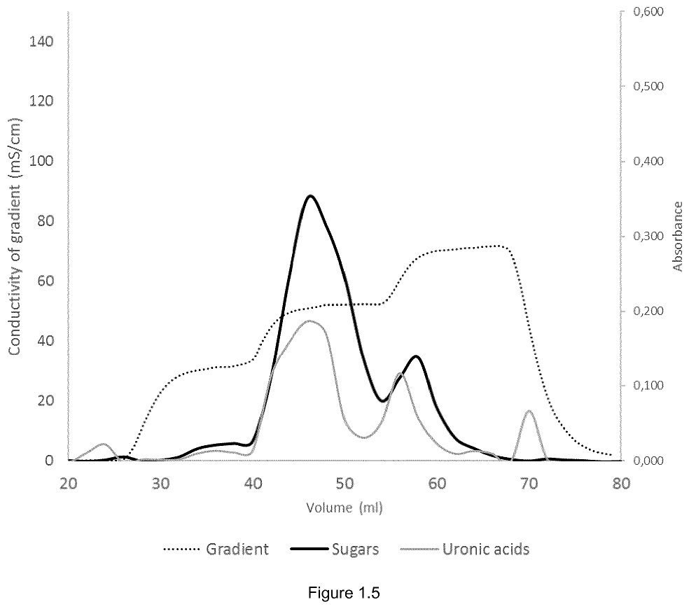

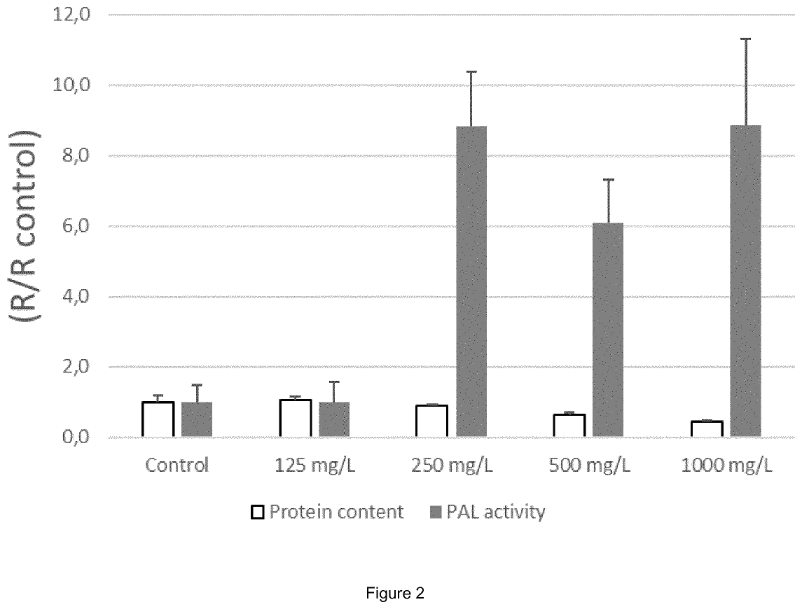

The (poly-anionic) saccharides are natural products (different from a synthetic polymer or oligomer), advantageously extracted from Spirulina genus biomass. They are acidic (poly-anionic) saccharides, preferably containing rhamnose, uronic acid and one or more sulphated groups and preferably of low viscosity in solution. As shown in FIG. 1.2, in these saccharides, rhamnose saccharide is identified as the major sugar residue, preferably representing more than 50%, preferably more than 55% by weight, more specifically between 55% and 60% by weight of the total (being 100% by weight) of the saccharides residues. Therefore, both fractions are obtainable from natural renewable source with little or no modifications.

Both fractions extracted from Spirulina genus, methanol-soluble and (poly) saccharides fractions, induce plant metabolic changes, in particular protein content variations, Phenylalanine Amnonia Lyase (PAL) activity increasing, proline accumulation and changes in the metabolites, advantageously triggering the induction of important plant defence reactions. The measure of the PAL activity, like the measure of the glucanase activity or of the chitinase activity is used to measure an elicitor effect of compounds applied upon plants and their capacity to trigger plant defence mechanisms against pathogen infections and aggression. In this way, an increase of the protein content of tomato leaves was observed 24 hours after foliar spraying with a solution of this Spirulina polysaccharides extracts.

Diverse application forms, in particular seed coating, soil amended, and plant roots dipping of these bioactive molecules and compositions comprising them were assessed in distinct plants species. However, treatment of whole plants, seeds, leaves, flowers, fruits and roots could be obtained by spraying, drenching, soaking, dipping, injection and via fertigation systems and means of the composition of the invention in dry form or as dilution of a solution comprising this composition. In divers crops used as plant models, the dose-response curves for both, methanol soluble and (poly) saccharides were established.

In divers experiments, chitosan saccharide, a well-known plant elicitor, has been used as positive control for comparative purpose. The inventors have discovered that another known elicitor, such as the chitosan saccharide and the Spirulina (poly) saccharides extract according to the invention can advantageously be used in combination (possibly through a stabilization of the applied polysaccharides) preferably under a suitable formulation, to enhance, in synergy, the characteristics of the applied (poly) saccharides according to the invention and extracted from Spirulina or to reduce the known side effects (toxicity) of the chitosan saccharides. Preferably, both compounds are used in synergy for improving plant biological activity, especially to increase plant growth or protein content in a plant as well as in other fields, including medical, pharmaceutical, neutraceutic, cosmetic, food, feed, beverage, industrial, including in textile and paper production, and/or environmental applications.

Preferably, the chitosan saccharide is either an oligosaccharide or a polysaccharide having a degree of acetylation lower than 50% and a degree of polymerization higher than 5 (five).

### Example 1: Extraction and Characterization of Bioactive Molecules from Spirulina Genus Biomass

1.1—Extraction in Alcohol Solution

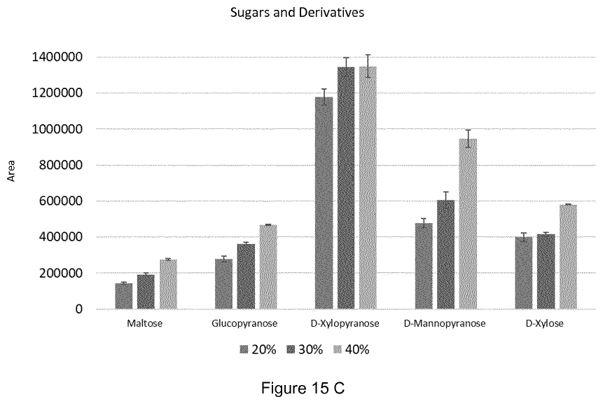

Commercial dried Spirulina genus biomass as described in ISBN 2-85744-853-X. (Ripley D. Fox. 1996. Editions Edisud, La Calade, R.N.7, 13090 Aix-en-Provence, France) and by Robert Henrikson (Apr. 20, 2011) AlgaelndustryMagazine.com) was subjected to lipid extraction as reported by Chaiklahan et al (Lipid and fatty acids extraction from the cyanobacterium Spirulina. Science Asia 34, 299. (2008)). Extraction was carried out in five time (20 minutes/each one) with a biomass-methanol ratio of 1:10 (w/v) at 25° C. After centrifugation at 4800×g for 10 minutes, the supernatant (Methanol soluble) was concentrate by rotatory evaporation. A similar extraction was also obtained with addition of different alcohol percentages, especially with different ethanol (ETOH) percentages (20% ETOH, 30% ETOH and 40% ETOH), as described in FIG. 15.

1.2—Extraction and Selective Precipitation of (Poly) Saccharides

Commercial dried Spirulina genus biomass 100 g, were added to 800 mL of water. The extraction process was performed in a single stage at 100° C. for 2 hours under continuous mixing at 120 rpm using a stirrer. After centrifugation at 4800×g for 10 minutes, the supernatant containing (poly) saccharides according to the invention was obtained.

(Poly) saccharides insoluble in CetylTrimethylAmmonium Bromide (CTAB): the (poly) saccharides according to the invention were precipitated with 1% (final concentration) CTAB solution. The precipitate was collected by centrifugation (10,000×g, 10 minutes) and washed stepwise with saturated sodium acetate in 95% ethanol, and absolute ethanol, respectively.

(Poly) saccharides insoluble in Ethanol: the (poly) saccharides were precipitated with 80% ethanol solution, which is the final concentration. The precipitate was collected by centrifugation (10,000×g, 10 minutes) and washed with absolute ethanol.

1.3—Characterization Methanol Soluble Fraction

1.3.1—GC-MS Analysis

GC-MS chromatogram of the MeOH soluble fraction obtained from Spirulina. GLC-MS analysis were carried out as follow: Dried samples were silylated by adding 10 μl of pyridine and 50 μl of N,O-Bis(trimethylsilyl)trifluoroacetamide (BSTFA) and heating for 30 minutes at 60° C. in sealed vials. The trimethylsilylated derivatives were separated by Gas Chromatography (GCMS-QP2010, Shimadzu) using a 0.25 mm×30 m optima 5 MS capillary column (0.25 μm film thickness) (Macherey Nagel, Germany) and identified by their electron impact (70 eV) mass data. He (0.75 ml min-1) was used as carrier gas. GLC conditions were as follows: initial column temperature 100° C., held for 6 minutes, ramped at 30° C. min-1 to 320° C. and held for 8 minutes; injector temperature 310° C., split ratio 20, 3.

1.3.2—XAD-Fractionation and Identification by GC-MS

A solution (2 mL) of methanol soluble from Spirulina biomass was applied to a XAD-column (25 mm×200 mm, BIO-RAD). The column was then eluted by using step elution with water, ethanol different concentrations and acetone at flow rate 2 mL/min. FIG. 1.1 A represents the GC-MS chromatograms of the different XAD fractions and shows that the alcohol extract comprises C16 and C18 fatty acids.

1.3.3—MS Analysis

MALDI-MS mass spectra were recorded using a Waters QToF Premier mass spectrometer equipped with a nitrogen laser, operating at 337 nm with a maximum output of 500 J.m-2 delivered to the sample in 4 nano-second pulses at 20 Hz repeating rate. Time-of-flight mass analyses were performed in the reflectron mode at a resolution of about 10,000. The samples were analyzed using dihydroxybenzoic acid/Dimethylaniline ionic liquid (DHB/DMA), that matrix was prepared as 25 mg in 250 μL acetonitrile:water (1:1) plus 5 μL of DMA. The matrix solution (1 μL) was applied to a stainless steel target and air dried. 1 μL aliquots of those samples were applied onto the target area already bearing the matrix crystals, and air-dried before adding 1 μL of Nal solution (2 mg/mL, acetonitrile:water). For the recording of the single-stage MS spectra, the quadrupole was set to pass all the ions of the distribution, and they were transmitted into the pusher region of the time-of-flight analyzer where they were mass-analyzed with 1 s integration time. Data were acquired in continuum mode until acceptable averaged data were obtained. FIG. 1.1B and FIG. 1.1C represent respectfully the MS spectra of the XAD fraction in ethanol and in acetone.

1.4—Characterization of (Poly) Saccharides

1.4.1—FT-IR Analysis

FT-IR analyses of spiruline polysaccharides was carried out by the potassium bromide (KBr) pellet method with a Perkin-Elmer Spectrum One FT-IR spectrometer (Norwalk, USA) in the range 400-4000 cm-1. FIG. 1.2 represent the FT-IR-Analysis spectra of the saccharides of the composition according to the invention.

1.4.2—TGA Analysis

Thermogravimetric analysis (Thermo-gravimetric analyser TGAQ500, from TA instruments) was used to examine the thermal stability against temperature of (poly) saccharides. Analyses were performed using around 20 mg sample in a nitrogen gas atmosphere. After a first step at 110° C. to evaporate the residual water, the (poly) saccharides were equilibrated at 40° C. Samples were finally heated from 40° C. to 600° C. at a heating rate of 3° C.min-1. These low heating rate was applied to reach a good resolution about the derivative weight percent. FIG. 1.3 represents the thermogravimetric analysis spectra of the saccharides composition according to the invention.

1.4.3—Viscosity

CTAB insoluble (poly) saccharide was dissolved in distilled water and solutions at different concentrations were prepared by diluting in water. In FIG. 1.4, the relative viscosity of the (poly) saccharide solutions in function of the final concentration was determined using a microviscometer Rheosense (http://www.rheosense.com/products/viscometers/microvisc/overview).

1.4.4—Composition Analysis

Monosaccharide composition was determined by hydrolysis of the (poly) saccharide samples (50 mg) with H2SO4 1 M at 100° C. for 3 hours. The hydrolyzed samples were converted to alditol acetates by successive NaBH4 reduction and acetylation with anhydride acetic in presence of 1-methylimidazole following the slightly modified method described by Blakeney et al, 1983 (A simple and rapid preparation of alditol acetates for monosaccharide analysis. Carbohyd. Res. 113, 291-299 (1983)).

The resulting alditol acetates were analyzed by gas chromatography-flame ionization detection (GC-FID) using a Shimadzu model GC-2010A gas chromatograph, using a DB-225 capillary column (30 m×0.25 mm i.d.), with helium as carrier gas. The analysis was carried out from 40 to 220° C. at 20° C. min-1, maintaining the temperature constant to the end of analysis (30 min). The monosaccharides (FIG. 1.2) were identified by their typical retention times.

The total sugars content of the spirulina polysaccharides is 44.1% as determined by the phenol-sulfuric and 6.5% in uronic acid content as determined according to the method of m-hydroxydiphenyl using glucuronic acid as the standard (Filisetti-Cozzi and Carpita, (Measurement of uronic acids without interference from neutral sugars. Analytical Biochemistry, 197, 157-162(1991)). The sulfate content is 11.7% as determined by the method of Jaques et al. 1968 (Jaques, L., Ballieux, R., Dietrich, C. & Kavanagh, L., 1968. A microelectrophoresis method for heparin. Canadian Journal of Physiology and Pharmacology, Issue 46, pp. 351-360).

1.4.5—Anion Exchange Chromatography

5 mg of (poly) saccharide of the invention was dissolved in 1 mL sodium acetate 0.02M at pH 5.5 and then applied to a Macro-Prep DEAE-column (10 mm×50 mm, BIO-RAD). The column was first eluted by sodium acetate 0.02 M at pH 5.5 and then by a step gradient of sodium acetate 0.02 M containing NaCl at pH 5.5 at flow rate 2 mL/min. 2 mL fractions as represented in FIG. 1.5 were collected and assayed by the phenol-sulfuric method for neutral hexoses according to Dubois et al (Colorimetric Method for Determination of Sugars and Related Substances. Anal Chem 28, 350-356 (1956)) and uronic acids content estimated as described before.

### Example 2: Induction of Plant Defense in Plant Cells Cultured In Vitro

The following Examples 2 to 7 present biological activity results obtained upon the induced plants and the Table 3 shows a summary of the biological trials.

Cell culture: Suspension-cultured cells of Arabidopsis thaliana strain L-MM1 ecotype Landsberg erecta were grown in Murashige and Skoog (Physiol. Plant, 15, 473-497 (1962)) medium (4.43 g/L) with sucrose (30 g/L) and 0.5 μg/mL of N-AcetylAsparatate (NAA) and 0.05 μg/mL of Kinetin, pH 5.7. Cultures were maintained under dark, at 24° C., on a rotary shaker at 100 rpm. Cells were diluted 10-fold in fresh medium every 7 days.

Bioassays: The (poly) saccharides were dissolved in distilled water, filtered through a 0.22 μm membrane filter (Millipore) and aseptically added to 5 mL of 7 days-old suspension-cultured cells and incubated 24 hours at 24° C. under mild agitation. The reaction mixture was centrifuged for 5 minutes at 100 g and 4° C. to collect the cells (PAL activity measurement).

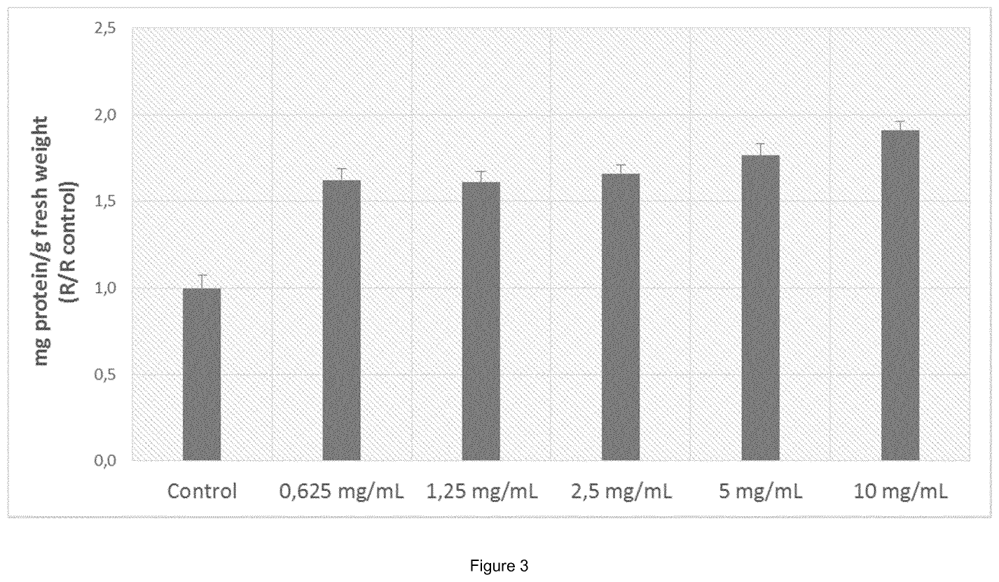

PAL activity: Cells were homogenized at 4° C. in 1 ml of 0.1 M borate buffer (at pH of 8.8) containing 2 mM mercaptoethanol. The homogenate was centrifuged at 4000 rpm for 10 minutes at 4° C. PhenylAlanine Ammonia lyase (PAL:EC 4.3.1.5) activity was determined in 0.125 ml supernatant in the presence of 1.37 ml 0.1 M borate buffer (at pH of 8.8) supplemented with 60 mM L-phenylalanine as described by Beaudoin-Eagan and Thorpe (Tyrosine and phenylalanine ammonia lyase activities during shoot initiation in tobacco callus cultures. Plant Physiology, 78(3), 438-441 (1985)). Protein concentration of the extracts was determined by the Bradford protein assay (Bio-Rad). FIG. 2 represent the protein content and PAL activity in Arabidopsis thaliana cell suspension, 24 hours after treatments with increasing concentrations of Spirulina saccharides.

### Example 3: Foliar Spraying of the Composition of the Invention on Tomato Plants

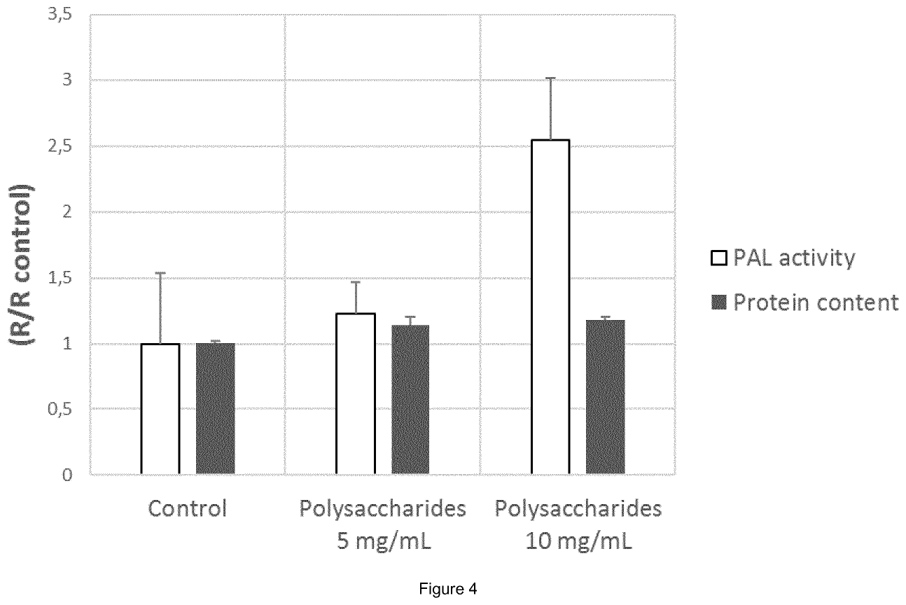

Tomato plants of the variety “Moneymaker” were cultivated in soil under controlled conditions with a light/dark regime of 16 hours/8 hours respectively, at 24° C., during 18 days before being sprayed with solutions containing the Spirulina (poly) saccharides. As a control, water containing about 0.01% Tween 20® was sprayed on the leaves. After 24 hours, the true leaves from plants treated by spraying were collected and ground in liquid nitrogen. Powdered leaves were extracted in 50 mM sodium acetate buffer at pH 5.2 containing about 5 mM EDTA, about 14 mM of beta-mercapto-ethanol and about 1.0 M NaCl to the rate of about 1 g of powdered leaves per 2 ml of buffer. FIG. 3 shows that foliar spraying of the composition according to the invention increases protein production in the treated plant.

### Example 4: Coating Wheat Seeds with (Poly) Saccharides of the Invention

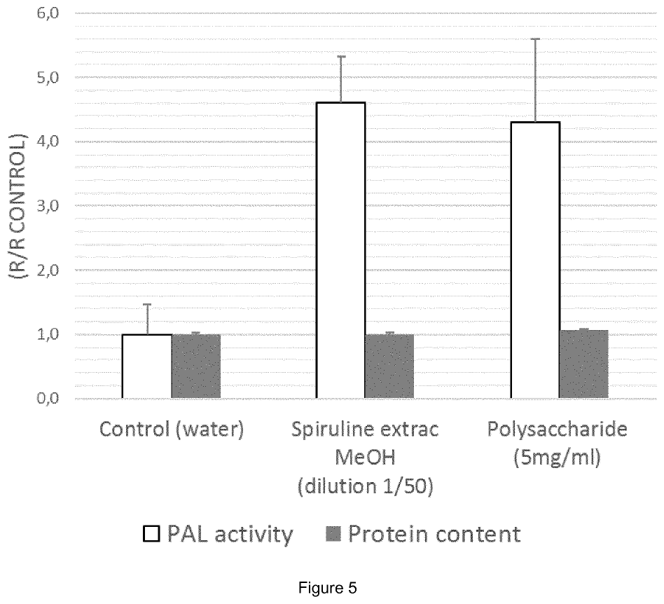

Seeds of wheat plant of the variety “Olivier” were coated with a solution containing increase concentration of Spirulina (poly) saccharides, and water (as control). After drying, 10 seeds of wheat per pots (3 pots per treatment) were planted and wheat plants growth for 2 weeks on soil under controlled conditions (light/dark regime of 16 hours/8 hours respectively, at 24° C.). FIG. 4 present the protein content and PAL activity in leaves of 10 days old wheat plants from seeds coated with increasing concentrations of Spirulina saccharides according to the invention.

### Example 5: Soil Amended with a Solution of (Poly) Saccharides of the Invention

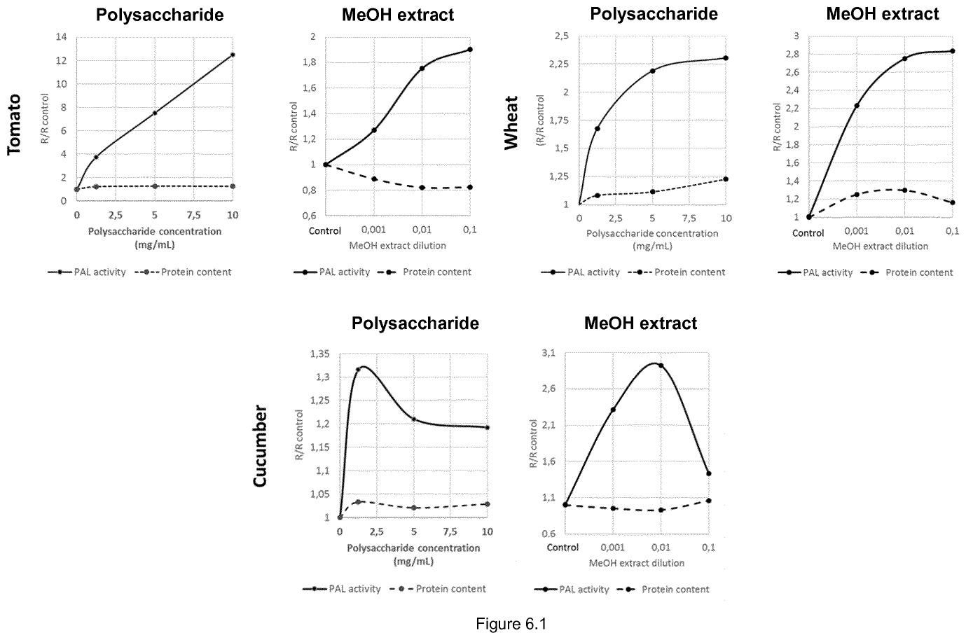

A solution of Spirulina (poly) saccharides according to the invention as well as the MEOH soluble extract according to the invention were added at the soil (ratio of solution to soil (1:1.2, v/w) before being added to planter pots. Wheat plants (variety “Olivier”) were cultivated in pre-treated soil under controlled conditions with a light/dark regime of 16 hours/8 hours respectively, at 24° C., during 15 days; 10 plants per repetition, 3 repetitions per treatment. True leaves from plants were collected and ground in liquid nitrogen. Powdered leaves were extracted in 50 mM sodium acetate buffer at pH 5.2 containing about 5 mM EDTA, about 14 mM .beta.-mercapto-ethanol and about 1.0 M NaCl to the rate of about 1 g of powdered leaves per 2 ml of buffer. FIG. 5 present protein content and PAL activity in leaves of 10 days old wheat plants growing on a soil pre-treated with a solution of Spirulina saccharides or methanol (MeOH) soluble extract.

### Example 6: Plant Roots Dipping

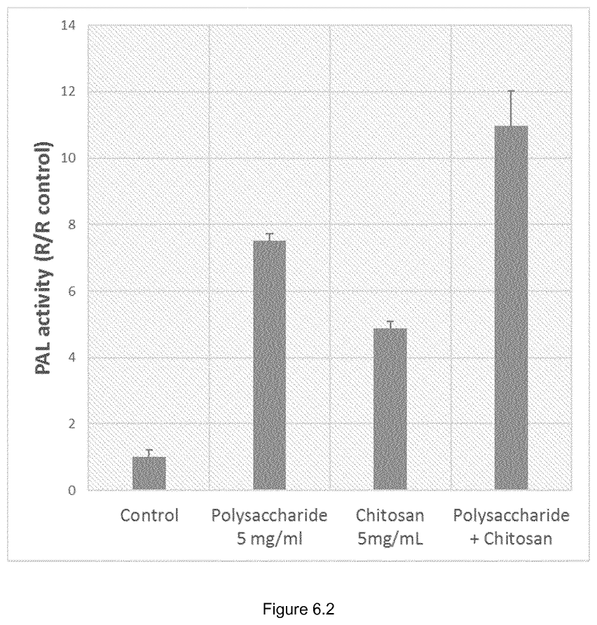

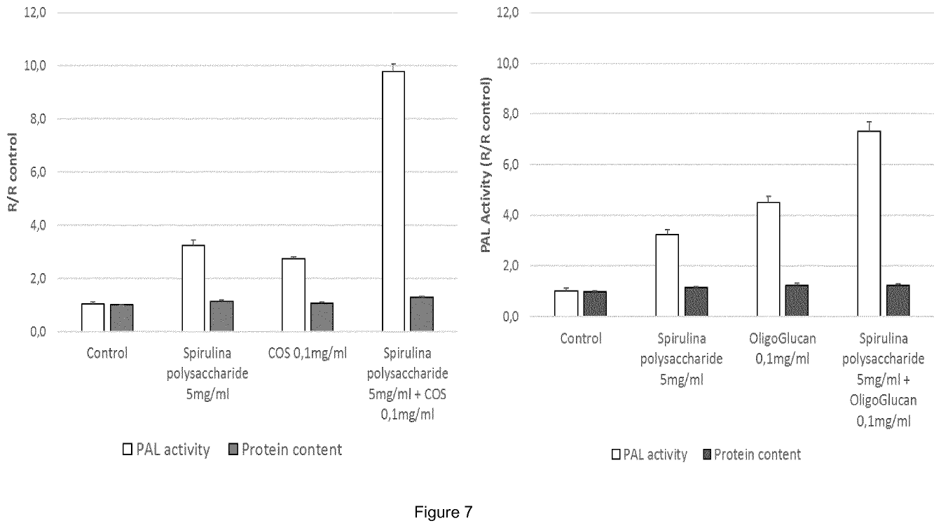

One week old seedlings of tomato (Lycopersicum esculentum), wheat and cucumber were carefully removed from the substrate and the roots immersed in a solution containing an increasing concentration of the Spirulina (poly) saccharide according to the invention, a solution of 5 mg/mL of chitosan saccharide or water being the Control for ½ hours. Then, the seedlings were planted and cultivated during 10 days in soil at 25° C. in a 16 hours day light/8 hours dark regime. The true leaves from plants treated were collected and ground in liquid nitrogen. Powdered leaves were extracted in 50 mM sodium acetate buffer at pH 5.2 containing about 5 mM EDTA, about 14 mM beta mercapto-ethanol and about 1.0 M NaCl to the rate of about 1 g of powdered leaves per 2 ml of buffer. FIG. 6.1 represents protein content and PAL activity in leaves of tomato, wheat and cucumber plants from seedling pretreated with increasing concentrations of the saccharides and increasing dilution of methanol (MeOH) extract according to the invention. FIG. 6.2 represents PAL activity in leaves of 10 days old tomato plants from seedling pre-treated by roots dipping with Spirulina saccharides, chitosan or a combination of both.

### Example 7: Induction of Proline Accumulation by Plant Roots Dipping

One week old seedlings of tomato plant (Lycopersicum esculentum) were carefully removed from the substrate and the roots immersed in a solution containing an increasing concentration of the Spirulina (poly) saccharide according to the invention, and water (Control) for ½ hours. The seedlings were planted and cultivated during 7 days in soil at 25° C. in a 16 hours day light/8 hours dark regime. The true leaves from plants treated were collected and ground in liquid nitrogen. The amino acid Proline (Pro) content was determined in tomato leaves according to Bates et al. (Rapid determination of free proline for water-stress studies. Plant and Soil 39, 205-207 (1973)) based on Proline's reaction with ninhydrin. The proline concentration was determined from a standard curve using proline (sigma) and expressed as relative values respect to the content in control treatment as R/R control.

Table 7 shows the amount of proline in tomato leaves a week after tomato roots dipping in different solution of Spirulina (poly) saccharides. Values of the mean+/−SE are reported (n=3). These results demonstrated the ability of Spirulina (poly) saccharides of the invention to induce advantageously the accumulation of proline in plants.

TABLE 7
Induction of proline accumulation proline in tomato
leaves a week after tomato roots dipping in different
solution of Spirulina (poly) saccharides

| | |
|---|---|
| Concentration of | Proline content |
| spirulina polysaccharides | (R/R control) |

| | | |
|---|---|---|
| 1.25 | mg/mL | 1.13 ± 0.02 |
| 5.00 | mg/mL | 1.22 ± 0.05 |
| 10.00 | mg/mL | 1.44 ± 0.05 |

### Example 8. Effects of Spirulina Polysaccharides in Combination with Other Elicitor Molecules Foliar Spraying on Tomato Plants

Tomato plants of the variety “moneymaker” were cultivated in soil under controlled conditions with a light/dark regime of 16 h/8 h respectively, at 24° C., during 18 days before being sprayed with solutions containing the spirulina polysaccharides (5 mg/mL), chitooligosaccharides (COS) (0.1 mg/mL), oligoglucan (0.1 mg/mL) or combination spirulina polysaccharides (5 mg/mL) and chitooligosaccharides (COS) (0.1 mg/mL) and spirulina polysaccharides (5 mg/mL) and oligoglucan (0.1 mg/mL). As a control, water was sprayed on the leaves.

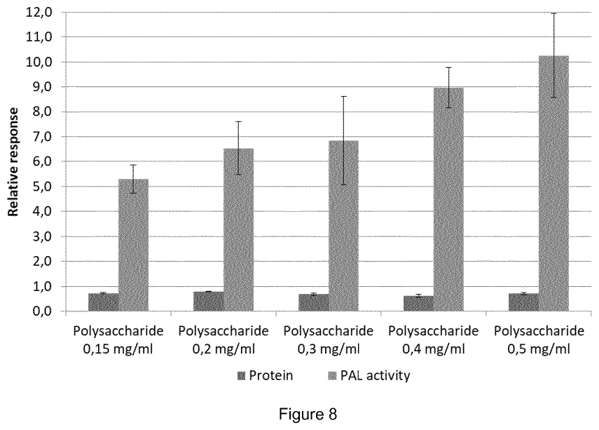

After 24 hours, the true leaves from plants treated by spraying were collected and ground in liquid nitrogen. Powdered leaves were extracted in 50 mM sodium acetate buffer pH 5.2 containing about 5 mM EDTA, about 14 mM beta-mercapto-ethanol and about 1.0 M NaCl to the rate of about 1 g of powdered leaves per 2 ml of buffer. Protein and PAL activity (FIG. 7) were determined as explained in previous examples.

### Example 9: Spirulan Polysaccharides Induce Plant Defense in Arabidopsis thaliana Cell Suspension

Spirulina polysaccharides fraction according to the invention was prepared as explained in example 1.

### Cell Culture:

Suspension-cultured cells of Arabidopsis thaliana strain L-MM1 ecotype Landsberg erecta were grown in Murashige T & Skoog F. (A revised medium for rapid growth and bioassays with tobacco tissue cultures. Physiol. Plant. 15:473-97, 1962)4.43 g/L) with sucrose (30 g/L) and 0.5 μg/mL of NAA and 0.05 μg/mL of Kinetin, pH 5.7. Cultures were maintained under dark, at 24° C., on a rotary shaker at 100 rpm. Cells were diluted 10-fold in fresh medium every 7 days.

### Bioassays:

The spirulina polysaccharides were dissolved in distilled water, filtered through a 0.22 μm membrane filter (Millipore) and aseptically added to 5 mL of 7 days-old suspension-cultured cells and incubated 24 hours at 24° C. under mild agitation. The reaction mixture was centrifuged for 5 min at 100 g and 4° C. to collect the cells (PAL activity measurement).
PAL activity:
Cells were homogenized at 4° C. in 1 ml of 0.1 M borate buffer (pH 8.8) containing 2 mM mercaptoethanol. The homogenate was centrifuged at 4000 rpm for 10 minutes at 4° C. PAL (EC 4.3.1.5) activity was determined in 0.125 ml supernatant in the presence of 1.37 ml 0.1 M borate buffer (pH 8.8) supplemented with 60 mM L-phenylalanine as described by Beaudoin-Eagan L D, Thorpe T A. (Tyrosine and Phenylalanine Ammonia Lyase Activities during Shoot Initiation in Tobacco Callus Cultures. Plant Physiol. 1985 July; 78(3):438-41). Protein concentration of the extracts was determined by the Bradford protein assay (Bio-Rad).

### Example 10: Induction of Plant Defense and Changes in Plant Metabolites Profiles after Foliar Spraying of Spirulina Polysaccharides

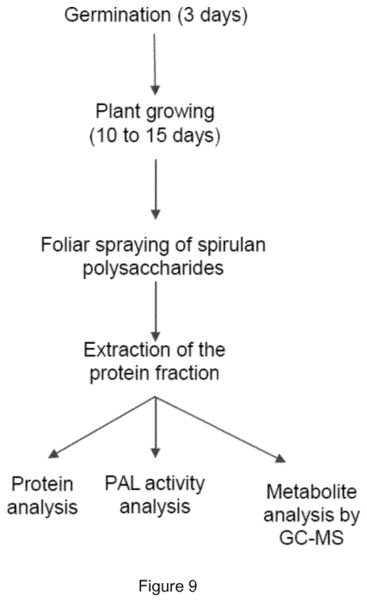

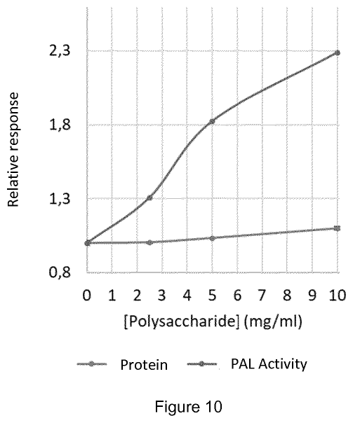

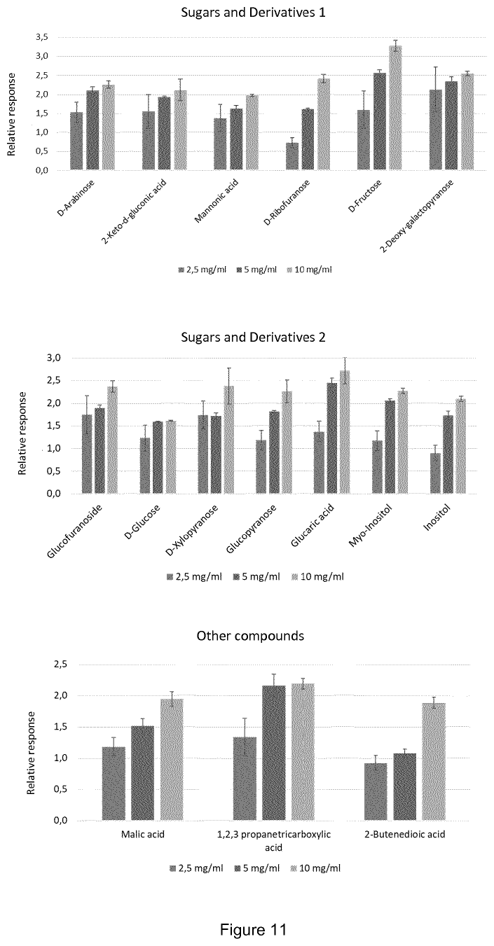

The inventors have conducted an experiment according to the method described here above and illustrated in the FIG. 9. The inventors have observed that changes in protein content and PAL activity are depending on spirulina polysaccharide concentration in the sprayed solution on tomato plants (see FIGS. 8 and 10)

This example 10 shows changes in different plant metabolites after foliar spraying of Spirulina polysaccharides. This means that Spirulina polysaccharide induce plant defense and also, changes in other plant metabolic pathways

### Example 11: Enzymatic Treatment of Spirulan Polysaccharides Enhances its Biological Activity

Preparation of low molecular weight Spirulan fractions obtained by enzymatic hydrolysis depends on the enzyme screening and selection, on the hydrolysis conditions, in particular the substrate/enzyme used, pH, temperature, hydrolysis time and on the fractionation of the enzymatic hydrolysis obtained products needed. Preferred conditions are the following: An efficient hydrolysis is obtained by addition of one or more hydrolysase enzymes, preferably selected from the group consisting of xylanases, cellulases or pectinases, active at suitable pH values, preferably between (about) 2 and (about) 5 or between (about) 3 and (about) 4, preferably at a pH of 3.5 and preferably at room temperature, preferably between (about) 20° C. and (about) 40° C., preferably at about 37° C. and for an adequate period, preferably after at least 10 hours hydrolysis time, but less than 2 or 3 days, preferably less than 1 day, to recover oligosaccharides and not monomers or dimers. The aim of this experiment was to compare biological activity before and after molecular weight reduction.

Spirulan at 5 g/l concentration was dissolved by overnight shaking at room temperature in 0.05 M citrate buffer pH 3.5 and 100 ml of the obtained Spirulan solution was mixed in 50 ml of a pectinase rich enzyme (Lyvelin) containing a highly active fungal endopolygalacturonase enzyme and was incubated at 37° c. for 24 hours. Spirulina polysaccharides fraction was prepared as explained in example 1 and spirulan hydrolysate was obtained by an enzyme addition to form a hydrolysis mix which is purified by ultrafiltration (UF) step to remove the added enzyme and form the so called “Spirulan hydrolysate” of the invention.

### Cell Culture:

Suspension-cultured cells of Arabidopsis thaliana strain L-MM1 ecotype Landsberg erecta were grown in Murashige and Skoog medium (4.43 g/L) with sucrose (30 g/L) and 0.5 μg/mL of NAA and 0.05 μg/mL of Kinetin, pH 5.7. Cultures were maintained under dark, at 24° C., on a rotary shaker at 100 rpm. Cells were diluted 10-fold in fresh medium every 7 days.

### Bioassays:

The spirulina polysaccharides were dissolved in distilled water, filtered through a 0.22 μm membrane filter (Millipore) and aseptically added to 5 mL of 7 days-old suspension-cultured cells and incubated 24 hours at 24° C. under mild agitation. The reaction mixture was centrifuged for 5 min at 100 g and 4° C. to collect the cells (PAL activity measurement).

### Pal Activity:

Cells were homogenized at 4° C. in 1 ml of 0.1 M borate buffer (pH 8.8) containing 2 mM mercaptoethanol. The homogenate was centrifuged at 4000 rpm for 10 minutes at 4° C. PAL (EC 4.3.1.5) activity was determined in 0.125 ml supernatant in the presence of 1.37 ml 0.1 M borate buffer (pH 8.8) supplemented with 60 mM L-phenylalanine as described by Beaudoin-Eagan and Thorpe (1985). Protein concentration of the extracts was determined by the Bradford protein assay (Bio-Rad).

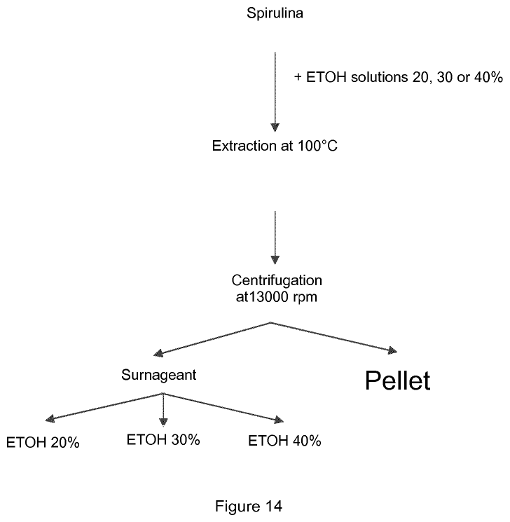

The anion-exchange chromatographic profile of the obtained low molecular weight spirulan (or obtained Spirulan hydrolysate) is presented in FIG. 13. According with this bioassay made upon Arabidopsis thaliana cell suspension and as shown in FIG. 13, reduction of spirulan MW (in the form of the spirulan hydrolysate) enhanced his biological activity in plants.

### Example 12: Characterization of ETOH Extracts from Spirulina

Here, we provide additional information on the general composition of various ETOH extracts from spirulina biomass and on the composition of some specific compounds (fatty acids, sugars, organic acids) in these ETOH extracts obtained from spirulina biomass. These are GC-MS analysis after TMS derivatization The following table 8 presents proteins, polyphenols and polysaccharides content of each ETOH fraction.

TABLE 8

| Component | 20% ETOH | 30% ETOH | 40% ETOH |
|---|---|---|---|
| Protein (mg/l) | 850 ± 30 | 760 ± 70 | 780 ± 30 |
| Polyphenols (mg/l) | 391 ± 20 | 448 ± 43 | 419 ± 19 |
| Polysaccharides (mg/l) | 4200 ± 200 | 3600 ± 150 | 2800 ± 150 |

One week old seedlings of tomato (Lycopersicum esculentum) were carefully removed from the substrate and the roots immersed in a solution containing ethanol extracts (20%, 30% and 40% ETOH extracts) from spirulina biomass or water (Control) for 0.5 hours. Then, the seedlings were planted and cultivated during 10 days in soil at 25° C. in a 16 hours daylight/8 hours dark regime. The true leaves from plants treated were collected and ground in liquid nitrogen. Powdered leaves were extracted in 50 mM sodium acetate buffer pH 5.2 containing about 5 mM EDTA, about 14 mM beta-mercapto-ethanol and about 1.0 M NaCl to the rate of about 1 g of powdered leaves per 2 ml of buffer. Applied alcohol extraction and analysis is presented in FIG. 15. The FIGS. 15A to 15C present the respective amounts of organic acids, fatty acids and saccharides present in each ETOH fraction.

### Example 13: Comparison of Plant Defense Induction (PAL Activity) of Different Ethanol Extracts from Spirulina Biomass

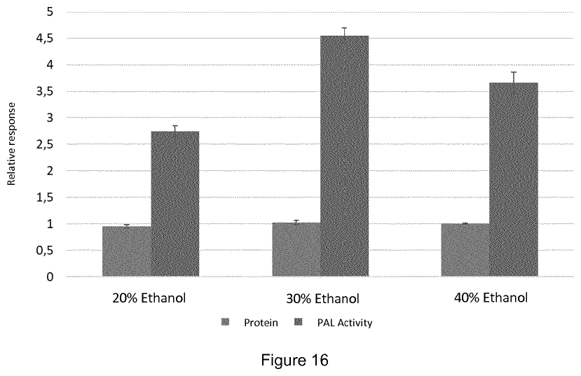

The FIG. 16 presents the measured PAL relative response according to the different added Spirulina ETOH extracts of the invention obtained with increased alcohol %, respectively 20% ethanol (Spirulina ETOH 20%), 30% ethanol (Spirulina ETOH 30%) and 40% ethanol (Spirulina ETOH 40%).

According with these results, Spirulina ETOH 30% extract are showing slightly higher bioactivity than other Ethanol (ETOH) extracts. Similar results were obtained with Methanol (METOH) extraction.

1. A composition comprising saccharides extracted from blue-green algae biomass, the said saccharides comprising
from 55% to 60% of rhamnose by weight,
from 6.5% to 10% of uronic acid by weight,
from 5% to 15% of sulfate groups by weight, and
less than 10% of xylose, glucose and galactose or a mixture thereof calculated by weight upon the total of the composition being 100%.

2. The composition according to claim 1, comprising saccharides extracted from genus Spirulina.

3. The composition according to claim 1, which is water soluble.

4. An enzymatic hydrolysate, preferably a pectinase hydrolysate of the composition according to claim 1.

5. The composition according to claim 1, which further comprises an alcohol soluble fraction extracted from genus Spirulina, which comprises beta-carotenes and fatty acids.

6. The composition according to claim 5, wherein the fatty acids are selected from the group consisting of C16 fatty acids, C18 fatty acids or a mixture thereof.

7. The composition according to claim 5, wherein the alcohol soluble fraction is an ethanol or methanol soluble fraction extracted from genus spirulina.

8. The composition according to claim 5, wherein the alcohol soluble fraction is a 30% ethanol or 30% methanol soluble fraction extracted from genus Spirulina.

9. The composition according to claim 5, wherein the alcohol soluble fraction comprises more than 440 mg/l polyphenols.

10. The composition according to claim 1, which further comprises a poly-cationic saccharide.

11. The composition of claim 10, wherein the poly-cationic saccharide is selected from the group consisting of a chitosan oligosaccharide, a functionally-modified chitosan oligosaccharide, chitosan polysaccharide, a functionally-modified chitosan polysaccharide, salt thereof, or combination thereof.

12. The composition of claim 11, wherein the chitosan or the functionally-modified chitosan has a degree of polymerization higher than 5 and a degree of acetylation lower than 50%.

13. The composition according to claim 1, which further comprises one or more of a saccharide, growth regulator or growth factor, vitamin, hormone, mineral, ion, nutriment, food additive, flavouring, colour, a phytosanitary product, phytonutrient, or combination thereof.

14. The composition according to claim 1, which is selected from the group consisting of a pharmaceutical composition, a veterinary composition, a nutraceutical composition, a food composition or feed composition including a functional food or a functional feed, a cosmetic composition, a phytosanitary product, an elicitor, a fertilizer, or an adjuvant of these products.

15. A biomaterial, preferably a textile, comprising the composition according to claim 1.

16. (canceled)

17. (canceled)

18. A method of inducing protection of a plant against infection by one or more of Phytophthora infestans, Phytophthora cinnamomi, Phytophthora capsici, Botrytis cinerea, Erwinia carotovora, Alternaria alternata, Alternaria solani, Fusarium spp., Erysiphe cichoracearum, Rhizoctonia solani or a combination thereof, the method comprising administering the composition of claim 1 to the plant.

19. A method for increasing protein content into a plant, the method comprising administrating the composition of claim 1 to the plant.

20. The method of claim 18, wherein the step of administrating the composition to the plant comprises foliar spraying of the composition upon the plant leaves, by coating the plant seeds with the composition, by dipping the plant roods with the composition or through amendment with the composition of the plant soil.

21. The method according to claim 19, wherein the composition is present in a solvent at a concentration higher than 0.1 mg/ml.

22. A method of treating wounds in a human or animal body comprising the step of administrating the composition of claim 1 to the human or animal body.
